(19) United States
(12) Patent Application Publication
(10) Pub. No.: US 2009/0030283 A1
(43) Pub. Date: Jan. 29, 2009

(54) ENDOSCOPE

(75) Inventors: Volkmar Freystein, Emmendingen (DE); Tim-Oliver Kern, Waiblingen (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: SCHOLLY FIBEROPTIC GMBH, Denzlingen (DE)

(21) Appl. No.: 12/170,708

(22) Filed: Jul. 10, 2008

(30) Foreign Application Priority Data
Jul. 11, 2007 (DE) 102007032201.3-51

(51) Int. Cl. A61B 1/07

(52) U.S. Cl. 600/182

(57) ABSTRACT

An endoscope (1) having a handle (5) and a probe (2) that can be connected in an interchangeable manner, with an imaging system (40) having an imaging optic (29) and an illumination device (32) arranged in the handle (5). The handle (5), at its distal end, has a coupling (4) for connection to the probe (2), and the probe (2) is interchangeable and provided with an image conducting fiber bundle (8) and a light conducting fiber bundle. The handle (5) is provided with a support end surface (27) in the coupling area, which has a predetermined distance from the imaging system (40) such that the image conducting fiber bundle (8) of the probe has a handle support end surface (27), planar and parallel in reference to the probe support end surface (12), that a clamping device is arranged at the coupling (4) to longitudinally position the probe (2) in reference to the handle (5) and in the clamped position of the coupling clamping device, the probe support end surface (12) is supported on the handle support end surface (27).

### CROSS-REFERENCE TO RELATED APPLICATIONS

This application claims the benefit of DE 10 2007 032 201.3-51, filed Jul. 11, 2007, which is incorporated by reference herein as if fully set forth.

### BACKGROUND

The invention relates to an endoscope with a handle and a probe that can be connected thereto in an exchangeable manner, with an imaging system with an imaging optic and an illumination device being arranged in the handle, with the handle being provided at its distal end with a coupling for connecting the probe and with the exchangeable probe being provided with an image conducting fiber bundle and a light conducting fiber bundle.

Endoscopes with interchangeable probes are known from prior art. For example, from the German patent publication DE 197 155 10 C2 an endoscope is known with a coupling device between the endoscope and a camera module, which allows a separation of the two parts. This coupling device is provided at one part with a circular groove and at the other part with a latch to engage this circular groove. The endoscope is provided with a focusing device having a collar for focusing, in order to allow focusing the imaging optic after the exchange of the endoscope and/or the camera module. For this purpose, the focusing device is provided with movable lenses, which require appropriate efforts and expenses. Furthermore, it is possible to accidentally misadjust the focus during operation.

### SUMMARY

Therefore the object of the invention is to provide an endoscope requiring no focusing when probes are exchanged and thus is easier and safer in its handling.

According to the invention this object is attained in that the handle is provided with a support end surface in the coupling area, which has a predetermined distance from the imaging system such that the image conducting fiber bundle of the probe is provided with a probe support end surface, planar and parallel in reference to the support end surface, at the coupling a clamping device is arranged for the longitudinal positioning of the probe in reference to the handle, and in the clamped position of the clamping device of the coupling, the probe support end surface is supported on the handle support end surface.

By having the clamping position of the probe support end surface supported on the handle support end surface, the probe is positioned in an unambiguously predetermined position in reference to the handle imaging system, particularly the imaging optic. Therefore, the probe can already be designed for the fixed focus point of the support end surface during its production, so that during the coupling of the probe to the handle no focusing is required. It is therefore possible to avoid a focusing device in the handle or the probe, thus rendering the production cheaper and more cost effective. Additionally, the exchange of probes can occur faster and easier and the focus cannot be accidentally misadjusted during operation. Through the use of the clamping device the coupling is locked and thus any accidental loosening of the probe is prevented.

In a preferred embodiment of the invention the coupling is provided with a push-in snapping connection, with preferably snapping hooks, being symmetrically arranged at the coupling, that engage a circular groove in the probe housing. In this way, the probe is already held at the handle during the axial connection process and then additionally positioned by the clamping device.

Preferably, at the exterior perimeter of the proximal end of the coupling, the clamping device is provided with a tensile thread, which engages a threaded bushing connected to the housing of the handle. In this manner, the coupling can be transferred into the clamped position by a simple rotation. For example, the thread is designed such that a quarter rotation is sufficient to reach the clamped position.

In a beneficial embodiment of the invention, a removable centering adapter is provided at the coupling, which aligns the probe in reference to the handle in a rotation-proof manner and after its removal considerably facilitates the cleaning of the inside of the handle. The centering adapter can here be produced as a cost-effective plastic part or be produced from another suitable material, as well.

The alignment of the support end surfaces is primarily designed for connecting the image conducting fiber bundle of the probe and the handle imaging system in order to omit focusing in the image transmission field. In order to ensure the precise attachment of the two support end surfaces of the image transmission, the handle illumination device is beneficially supported in a movable fashion in the longitudinal direction and compressed against the probe support end surface by a spring. The off-set potentially existing in the separating area can be compensated in this way and has no effect on the precise contact of the image transmitting support end surfaces.

In another embodiment of the invention, the probe is provided with a socket for the image conducting fiber bundle to accept the proximal end of the image conducting fiber bundle of the probe, and the proximal end of the socket for the image conducting fiber bundle is supported planar and parallel in reference to the support end surface of the image conducting fiber bundle of the probe. The socket of the image conducting fiber bundle increases the support surface of the probe support end surface and thus improves the planarity in reference to the handle support end surface. Furthermore, the image conducting fiber bundle can only be cut planar at the end surface of the socket of the image conducting fiber bundle together with the light conducting fiber bundle after the insertion into the socket for the image conducting fiber bundle such that planarity is also optimized, here.

Additionally, by having the planar support end surfaces, the end surfaces of the image and light conducting fiber bundle are easier to clean, because the entire support end surface can be wiped off.

In a particularly beneficial embodiment, the imaging system is provided in the radiation path of the imaging optic with an imaging sensor, which directly transfers the image of the probe into electric signals. Of course, instead of the image sensor it is also possible to provide an eye-piece for direct observation.

Further advantageous embodiments of the invention are discernible from the description that follows and the drawings or from combinations of the individual features.

### BRIEF DESCRIPTION OF THE DRAWINGS

A preferred embodiment of the invention is explained in greater detail using the drawings. Shown are:

FIG. 1 is a perspective explosive view of an endoscope,

FIG. 2 is a cross-sectional view of the parts of an interchangeable probe for an endoscope,

FIG. 3 is a partially cross-sectional view of the handle with a coupling and a centering device in a disassembled state,

FIG. 4 is a partially cross-sectioned view of an assembled endoscope,

FIG. 5 is an enlarged detailed view in the separating area between the handle and the probe, and

FIG. 6 is an exploded view of the coupling parts.

### DETAILED DESCRIPTION OF THE PREFERRED EMBODIMENTS

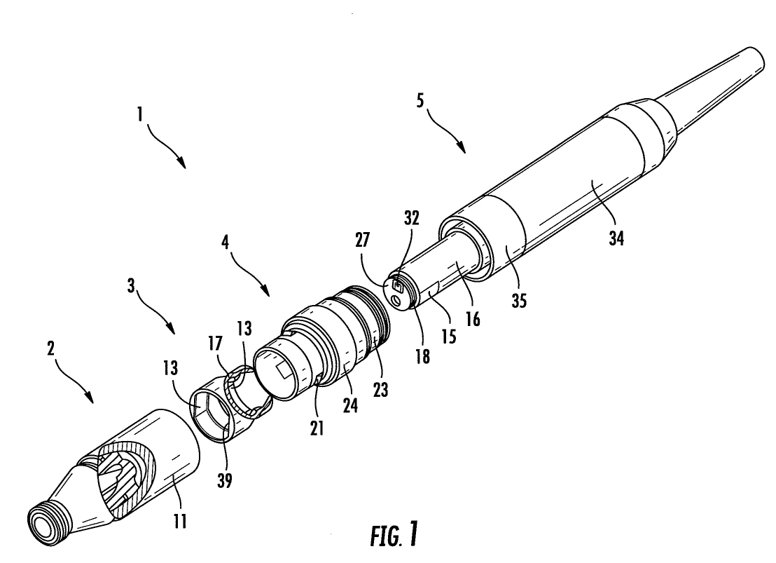

FIG. 1 shows an endoscope, marked in its entirety as 1. The endoscope 1 is essentially divided into four functional parts: an interchangeable probe 2, a centering adapter 3, a coupling 4, and a handle 5.

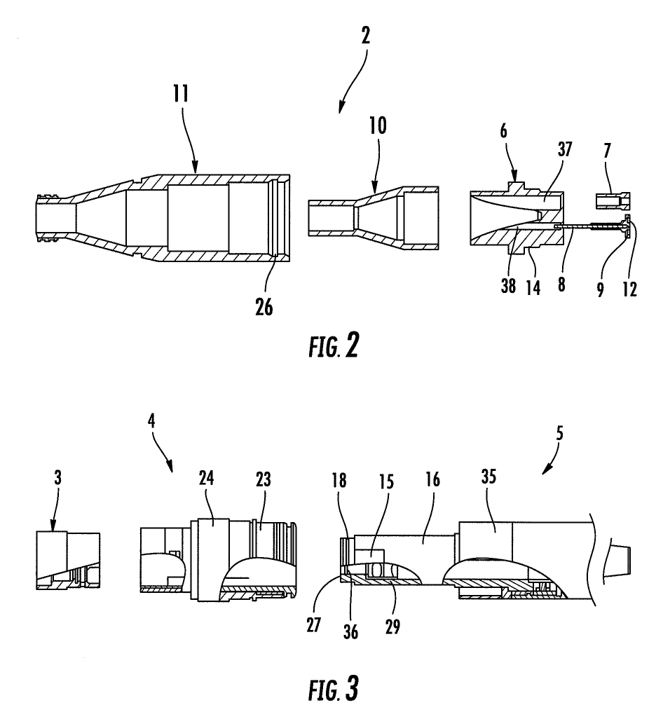

As discernible from FIG. 2, the probe 2 is provided with an inner support part 6, in which receiving bores 37, 38 are arranged for a light conducting fiber bundle and an image conducting fiber bundle 8. The light conducting fiber bundle (not shown) is held in a socket 7 for the light conducting fiber bundle, while the image conducting fiber bundle 8 is guided in the socket 9 for the image conducting fiber bundle. The inner support part 6 is inserted into an inner sheath 10 and together therewith forms a probe unit which can be inserted into a probe housing 11.

An essential advantage of the probe unit is the fact that it can be processed prior to being inserted into the probe housing 11. In this way it is possible to face grind the light and the image conducting fiber bundle 8 together with the light conducting fiber bundle socket 7 and the image conducting fiber bundle socket 9 in order to yield a probe support end surface 12 with good surface characteristics and planarity in this manner, which cannot be achieved by processing the individual parts. The socket 9 for the image conducting fiber bundle is provided at the face with a circular groove, into which a soft-elastic sealing means can be inserted, additionally preventing the interspersing of light from the light conducting fiber bundle into the image conducting fiber bundle and thus to prevent any reduction of the contrast conditions.

The centering adapter 3, such as shown in FIG. 1 and FIG. 2, is provided at both ends with differently long centering surfaces 13, 39 which engage the respective centering surfaces 14, 15 at the interior part 6 of the probe and at the object carrier 16 of the handle. This ensures that the probe 2 and the handle 5 can be connected in their correct position and in a rotation-proof manner. At the interior perimeter of the end of the centering adapter facing the handle, an O-ring 17 is arranged in an encircling groove which, when the centering adapter 3 is placed on the handle 5, engages a corresponding groove 18 at the exterior perimeter of the distal end of the optic carrier 16 such that the centering adapter 3 is held on the handle 5 and is secured from an unintended removal, however it can be removed by way of a stronger pulling.

The handle 5 and the probe 2 are mechanically connected by the coupling 4 in a fixed manner. The coupling 4 is shown in detail in FIG. 6. Three spring arms 20 are arranged at the interior part 19 of the coupling, symmetrically over the circumference, each of which are provided at their free ends with a wedge-shaped snapping hook 21. An exterior thread 22 is arranged at the exterior circumference of the proximal end of the interior piece 19 of the coupling, onto which the threaded socket 23 can be screwed having an internal thread. In the area of the spring arms 20, a longitudinal mobile external coupling piece 24 can be placed coaxially onto the internal coupling piece 19, provided with openings 25 adjusted to the snapping hooks 21, through which the snapping hooks 21 protrude outwardly, with the spring arms 20 being relaxed.

When introducing the coupling 4 into the probe 2, the snapping hooks 21 engage an encircling groove 26 at the interior perimeter of the housing 11 of the probe (FIG. 2). Due to the wedge shape of the snapping hooks 21, it is impossible to simply pull off the probe 2.

In order to loosen the coupling 4 the external coupling piece 24 is moved in the direction of the handle 5. In this way, the openings 25 slide along the diagonal end surfaces of the snapping hooks 21 and press them radially inwardly such that the snapping hooks 21 are positioned flush in reference to the exterior perimeter of the exterior coupling piece 24 and thus the connection to the probe groove 26 is loosened. This unlocking motion against the direction of separation is advantageous because an accidental loosening is impossible.

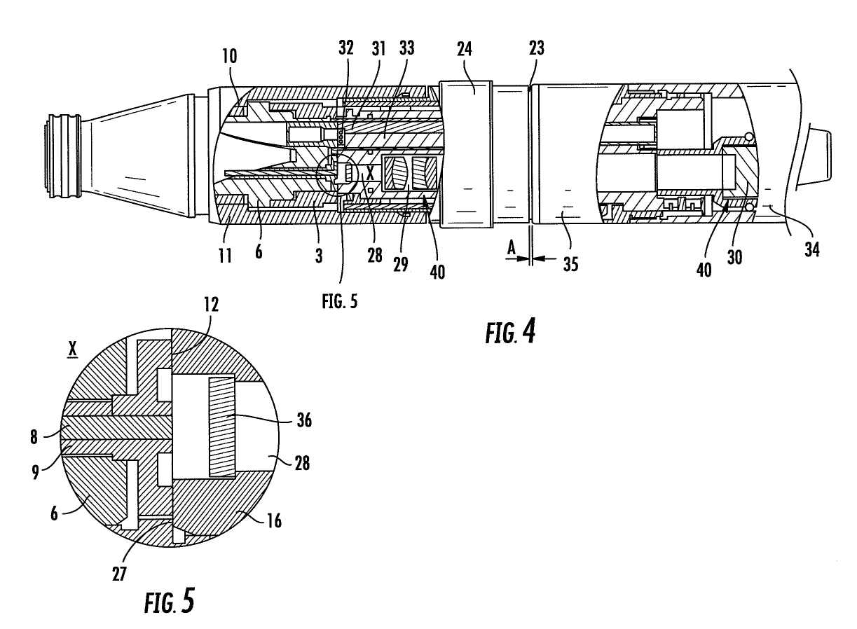

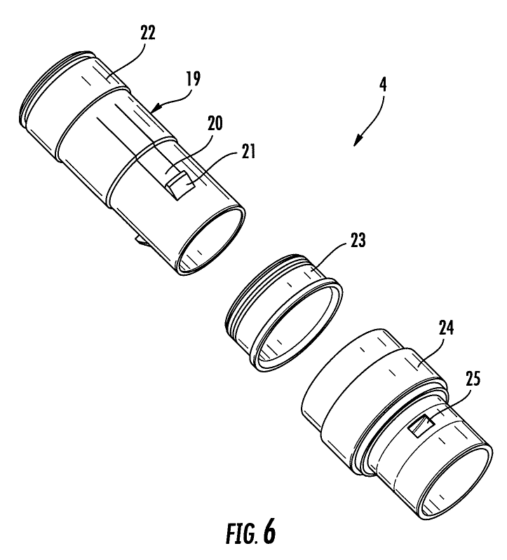

The handle 5 is shown in FIGS. 3 and 4 in a partially cross-sectional illustration in order to clearly show the interior design. At its distal end, the object carrier 16 has an even facial surface 27, which serves as a support end surface 27 for the probe support end surface 12 (FIG. 5). A longitudinal bore 28 is arranged in the object carrier 16, into which a plane face glass 36 is inserted in the proximity of the support end surface 27. The imaging optic 29 is located in the bore 28, in the rear connector at the plane face glass 36, which in the exemplary embodiment comprises two lenses and an image of the end surface of the probe image conducting fiber bundle 8, which is radiated on the support end surface 27, displayed on the image sensor 30 located at the end of the bore 28. The imaging optic 29 and the image sensor 30 form the imaging system 40 in the example shown. Of course, it is also possible to use an eye-piece instead of the image sensor 30 for directly observing the image or to forward the image to an external image sensor by another image conducting fiber bundle.

In another longitudinal bore 31 in the object carrier 16 there is an illumination source, for example comprising a light diode 32. The light diode 32 is mounted on a carrier element 33, supported to be movable in the longitudinal direction and rod-shaped. The carrier element 33 is pressed by a spring in the direction towards the socket for the light conducting fiber bundle 7 of the probe in order to ensure good light transmission. Due to this, even in case of a potentially existing off-set between the image transmitting support end surfaces and the light transmitting end surfaces, it is ensured that the probe support end surface 12 is precisely supported on the handle support end surface 27. Instead of the light diode a light conducting fiber bundle may also be provided and perhaps be guided out of the handle and connected to an external illumination source.

The coupling 4 is pushed coaxially onto the optic carrier 16 until the threaded socket 23 engages the front part 35 of the handle, namely by way of a press fit. However it is also possible to fasten the threaded socket 23 via an external thread into the internal thread at the front part 35 of the handle. The internal coupling piece 19 and the external coupling piece 24 are now rotational in the threaded socket 23 and movable via the tensile thread 22 in the longitudinal direction.

Similar to the probe 2, the object carrier 16 can be completely provided with all components before it is installed in a front part 35 of the handle.

After the probe 2 has been plugged onto the coupling 4, the probe 2 is held by the snapping plug-in connection and then the coupling can be stressed by way of rotation at the external coupling part 24. By rotating the tensile thread 22, the internal coupling part 19 and thus also the probe 2 engaged in the snapping hooks 21 is pulled in the longitudinal direction towards the handle 5, until the probe support end surface 12 is supported on the handle support end surface 27. In this way, the alignment of the image conducting fiber bundle 8 of the probe to the focus point of the imaging optic 29 is ensured and is always constant so that focusing is no longer necessary.

Additionally, for tightening the coupling 4 by the tensile thread 22, the distance A between the external coupling part 24 and the threaded socket 23 is so small that any loosening of the snap hook-connection between the probe 2 and the coupling 4 is no longer possible.

1. An endoscope (1) comprising a handle (5) and a probe (2) that can be connected thereto in an interchangeable manner, with an imaging system (40) being arranged in the handle (5) having an imaging optic (29) and an illumination device (32), the handle (5) being provided at a distal end thereof with a coupling (4) for connecting the probe (2), and with the probe (2) being interchangeable and including an image conducting fiber bundle (8) and a light conducting fiber bundle, the handle (5) is provided in a coupling area with a support end surface (27) having a predetermined distance in reference to the imaging system (40), the image conducting fiber bundle (8) of the probe is provided with a probe support end surface (12) that is planar and parallel in reference to the handle support end surface (27), a clamping device is provided at the coupling (4) for a longitudinal positioning of the probe (2) in reference to the handle (5), and in the clamped position of the coupling clamping device, the probe support end surface (12) is supported on the handle support end surface (27).

2. An endoscope according to claim 1, wherein the coupling (4) comprises a plug-in snap connection with symmetrically arranged spring hooks (21), which engage a circular groove (26) in a housing (11) of the probe.

3. An endoscope according to claim 1, the clamping device is provided at an external perimeter of a proximal end of the coupling (4) with a clamping thread (22) engaging a threaded socket (23) connected to a housing (34) of the handle.

4. An endoscope according to claim 1, characterized in that a centering adapter (3) is provided between the probe (2) and the handle (5) that can be inserted for a rotation-proof alignment of the probe (2) in reference to the handle (5).

5. An endoscope according to claim 1, wherein the illumination device (32) is located in the handle in a longitudinally movable fashion and is held in contact with the light conducting fiber bundle of the probe by a spring.

6. An endoscope according to claim 1, wherein the probe (2) is provided with an image conducting fiber bundle socket (9) to accept the proximal end of an image conducting fiber bundle (8) of the probe and the proximal end of the socket (9) of the image conducting fiber bundle is provided in a planar and parallel position in reference to the support end surface (12) of the image conducting fiber bundle (8) of the probe.

7. An endoscope according to claim 1, wherein a handle imaging system (40) is provided with an image receiver (30) having a predetermined distance in reference to a handle imaging optic (29), and comprises a CCD or CMOS image sensor.

8. An endoscope according to claim 2, wherein in the clamped position of the coupling clamping device, a distance (A) between the coupling exterior (24) and the threaded socket (23) is smaller than a connection path of the plug-in snap connection.
